(19) United States
(12) Patent Application Publication
(10) Pub. No.: US 2023/0417539 A1
(43) Pub. Date: Dec. 28, 2023

(54) MACHINE OF PASTE SHRINKAGE AND EXPANSION IN Z DIRECTION

(71) Applicant: Henkel AG & Co. KGaA, Duesseldorf (DE)

(72) Inventors: Wei Xiong, Shanghai (CN); Carol XiaoChuan Liu, Shanghai (CN); Jing Zhou, Shanghai (CN)

(21) Appl. No.: 18/244,576

(22) Filed: Sep. 11, 2023

Related U.S. Application Data
(63) Continuation of application No. PCT/CN2021/079888, filed on Mar. 10, 2021.

(51) Int. Cl. G01B 11/06

(52) U.S. Cl. CPC G01B 11/0625

(57) ABSTRACT

The disclosed provides a machine of paste shrinkage and expansion for timely monitoring a paste shrinkage in a Z direction during a UV curing process and for obtaining a change rate of a paste in a Z direction after a certain process. The machine comprises: a bottom substrate; a top substrate which is transparent and configured to be placed on the paste so that the paste is sandwiched between a top surface of the bottom substrate and a bottom surface of the top substrate, wherein the top surface of the bottom substrate and the bottom surface of the top substrate are provided with first and second reflecting layers, respectively, and define a thickness of the paste; a platform for supporting the bottom substrate; a plurality of pressing clamps configured for securing the bottom substrate on the platform in the Z direction; a plurality of pushing clamps configured for securing the bottom substrate in radial and circumferential directions; a plurality of UV sources positioned uniformly around the paste; and a sensor configured for measuring the thickness of the paste. The bottom substrate and the platform comprise a substrate dial and a platform dial, respectively, and the bottom substrate has a profile consistent with the platform dial. The disclosed provides methods for timely monitoring a paste shrinkage in a Z direction during a UV curing process and for obtaining a change rate of a paste in a Z direction after a certain process.

### TECHNICAL FIELD

The disclosure relates to a machine of paste shrinkage and expansion in a Z direction. In particular, using the machine, the paste can be UV cured and a paste shrinkage in the Z direction during the UV curing process can be timely monitored, and a change rate of the paste in the Z direction can be obtained after a certain process which can be the UV curing process, a thermal curing process or a reliability test process. A method for timely monitoring the paste shrinkage in the Z direction during the UV curing process and a method for obtaining the change rate of the paste in the Z direction after the certain process are also provided.

### BACKGROUND ART

Adhesives are widely used in many technical fields. For example, an active alignment (AA) adhesive can be used in AA process in compact camera modules (CCM) and Optical module film. Shrinkage and expansion of the adhesive substantially affect a lens of the CCM, and therefore it is important to know rules of the shrinkage and expansion of the adhesive during curing process.

In general, a volume and linear shrinkage of plastic materials, unsaturated polyester and epoxy resin can be measured by standard ISO 352, and measuring the volume and linear shrinkage is a good way to find out the rules of the shrinkage and expansion of the adhesive. However, this method cannot be totally aligned with real applications regarding the shrinkage and expansion in a Z direction for some factors, for example, due to resin out-gas during thermal curing process, especial for UV curable adhesive paste. Semicircle groove method is used by ASTM D2566-79 standard, which can't be applied to UV curable adhesive paste due to heterogeneous curing and the change of resin coefficient of thermal expansion for state change. The heterogeneous curing issue is then be solved using a cylinder method. However, a heterogeneous cavity is generated for cylinder method, which affects the accuracy of data. Meanwhile, many companies use a machine of 3D Dimensional Laser Confocal and a Vernier Caliper to measure a dimension of the paste before and after curing, but the accuracy will be affected by noise at a measuring position for each measurement.

It is desired to solve the problems as above.

### SUMMARY OF THE INVENTION

An object of the disclosure is to solve one or more of the above problems.

The object is achieved by a machine of paste shrinkage and expansion for timely monitoring a paste shrinkage in a Z direction during a UV curing process in claim 1, a method for timely monitoring a paste shrinkage in a Z direction during a UV curing process using the machine of paste shrinkage and expansion in claim 11, a machine of paste shrinkage and expansion for obtaining a change rate of a paste in a Z direction after a certain process in claim 15, and a method for obtaining a change rate of a paste in a Z direction after a certain process using the machine of paste shrinkage and expansion in claim 16.

With the machine and the method described in the disclosure, the paste shrinkage over UV applied time in the Z direction during the UV curing process can be timely monitored, which facilitates to analyze the shrinkage trend or the presence of the shrinkage delay during the UV curing process. This also provides a great significance to understand, control or fully make use of the curing kinetic of UV curing. Further, the paste's UV response performance for difference compositions can be detected using the machine and the method described above.

In addition, the change rates of the paste in the Z direction after the UV curing process, the thermal curing process and the reliability test process can be obtained. For an adhesive used in a camera, this helps to simulate a focal length change of the camera after AA process and reliability test.

### BRIEF DESCRIPTION OF THE DRAWINGS

The principle and various aspects of the present disclosure will be well understood from the following description in conjunction with the attached drawings. The drawings are not on scales, and they are given only for illustration and explanation purpose. Components or elements which are shown in the drawings are not necessary to be included in all embodiments of the disclosure and components or elements which are not shown in the drawings may be present in some embodiments of the disclosure. In the drawings:

FIG. 1 is an illustrative view of a machine of paste shrinkage and expansion in a Z direction constructed according to the principle of the disclosure;

FIG. 2 illustrates a portion of the machine in FIG. 1;

FIG. 3 illustrates a portion of the machine in FIG. 1;

FIG. 4 illustrates a portion of the machine in FIG. 1;

FIG. 5 illustrates a portion of the machine in FIG. 1;

FIG. 6 illustrates a portion of the machine in FIG. 1;

FIG. 7 illustrates the principle how a sensor of the machine measures a thickness of a paste;

FIGS. 8a-8d show alignment features on a bottom substrate and a platform of the machine and illustrate how they cooperate with each other;

FIG. 9 is a flow chart of a method for timely monitoring a paste shrinkage in a Z direction during a UV curing process;

FIG. 10 is a flow chart of a method for obtaining a change rate of the paste in the Z direction after a certain process;

FIGS. 11a-c are sketches showing a thickness change of three kinds of different adhesive pastes in the Z direction over UV applied time during a UV curing process, respectively, according to a first example;

FIG. 11d is a comparison of the change rate of the three adhesive pastes in the Z direction after the UV curing process;

FIGS. 12 and 13 are comparisons of the change rate of the three adhesive pastes in the Z direction after a thermal curing process and a reliability test process, respectively; and

FIGS. 14a-14b are sketches showing a thickness change of two kinds of different adhesive pastes in the Z direction over UV applied time during a UV curing process, respectively, according to a second example.

### DETAILED DESCRIPTION OF PREFERRED EMBODIMENTS

Throughout the drawings, like reference numerals refer to like components throughout this specification. This specification does not describe all components of the embodiments, and general information in the technical field to which the present disclosure belongs or overlapping information between the embodiments will not be described.

It will be understood that the terms “includes”, “comprises”, “including” and “comprising” when used in this specification specify the presence of a stated component, but do not preclude the presence or addition of one or more other components. Also, it is to be understood that the singular forms “a” “an” and “the” include plural referents unless otherwise stated. A component which is described or shown as being a unitary component may be implemented as including a plurality of components and an assembly or portion which is described or shown as including a plurality of components may be implemented as a unitary component unless the context clearly dictates otherwise.

In general, the disclosure relates to a machine of paste shrinkage and expansion in a Z direction, and the machine can provide two functions. The paste can be cured by a UV light in this machine and a first one of the functions is to monitor a paste shrinkage in the Z direction over UV applied time during this UV curing process, and a second one of the functions is to facilitate to obtain a change rate of the paste in the Z direction after a certain process.

In particular, in this specification, the paste is a kind of adhesive or adhesive sample in a form of paste, and may be referred to as “adhesive paste” for convenience. The term “certain process” can be any process the paste may be subjected to during which the paste may shrink or expand due to some special composition(s) contained in the paste. For example, the certain process may be the UV curing process mentioned above during which the paste will be cured using the UV light due to a UV curing agent contained in the paste, the certain process may be a thermal curing process during which the paste will be cured, in a thermal oven for example, due to a thermal curing agent contained in the paste, or the certain process may be a reliability test process during which the paste will be put into a box of high temperature and high humidity (HTHH). In an example to be described, the thermal curing process is carried out after the UV curing process and the reliability test process is carried out after UV curing and the thermal curing process. However, this is not necessary and the three processes can be carried out independently or in any combination.

The paste may shrink or expand depending on actual compositions contained the paste. For example, three kinds of adhesive pastes used in the example to be described in this specification shrink during the UV and thermal curing processes and expand during the reliability test process. To identify the degree of shrinkage or expansion of the paste after the certain process, a term “change rate” is used in the specification which means a shrinkage rate if the paste shrinks or an expansion rate if the paste expands. In the machine, the paste is dispensed in an annular pattern on a bottom substrate of the machine which is substantially arranged in a horizontal plane, the Z direction is perpendicular to the plane and to the bottom substrate, and the change rate of the paste in the Z direction means the degree of the change of a thickness of the paste in the Z direction. The thickness of the adhesive paste can also be referred to as a bonding line thickness in related technical fields and is denoted by a reference sign B in the specification.

In general, the change rate R of the paste in the Z direction after the certain process is calculated out based on an initial thickness value of the paste in the Z direction before the certain process, Bᵢₙᵢ, and a final thickness value of the paste after the certain process, B_{fin}, using the formula I: R=(Bᵢₙᵢ−B_{fin})/Bᵢₙᵢ. The thickness values Bᵢₙᵢ and B_{fin} can be the thickness values measured at one and the same point of the paste before and after the certain process. However, in order to improve the precision, the thickness values Bᵢₙᵢ and B_{fin} can be averaged thickness values based on many thickness values measured at a set of points (preferably, one and the same set of points) in a circumferential direction of the pattern of the paste before and after the certain process, respectively. This can be achieved using the machine and method to be described in this specification.

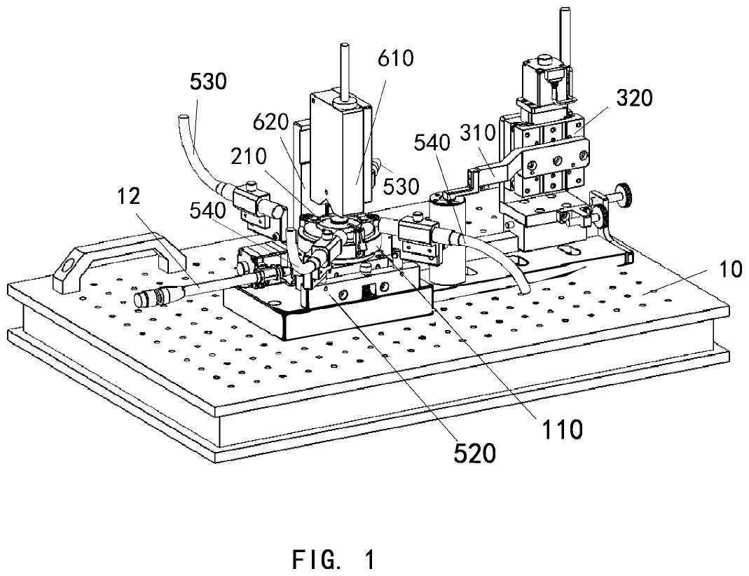

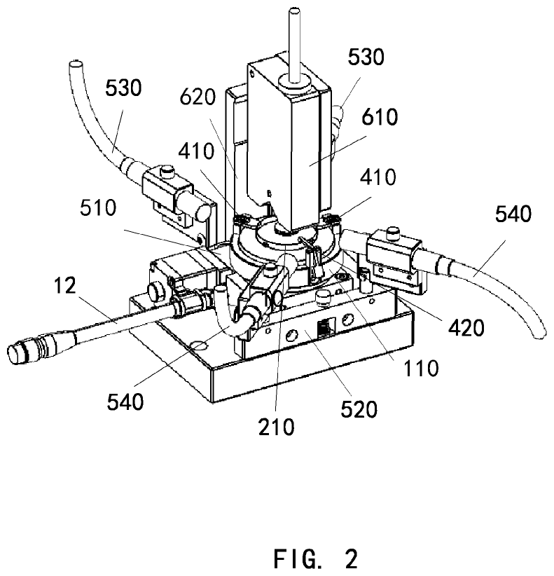

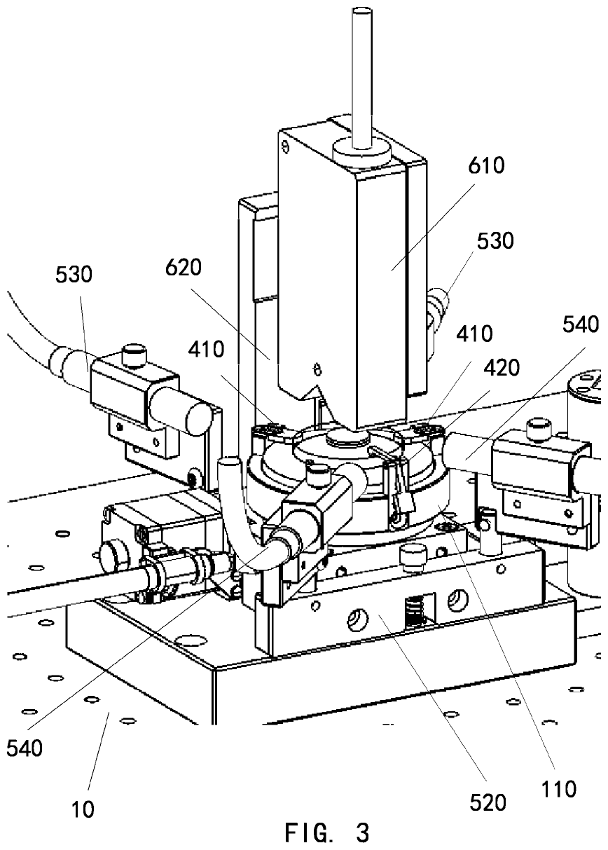

The disclosure will be described in detail with reference to the drawings. FIGS. 1-6 illustrate a machine of paste shrinkage and expansion in a Z direction constructed according to the principle of the disclosure, in which FIG. 1 is an overall view of the machine and FIGS. 2-6 each are enlarged views illustrating a portion of the machine in FIG. 1.

The machine of paste shrinkage and expansion in the Z direction can be placed on a table 10 or similar component, and comprises a rotatable platform 110 which can be mounted on the table 10. A drive mechanism (not shown) is provided for the platform 110 and configured for driving and rotating the platform 110. The drive mechanism can be any well-known drive mechanism in the art and, for example, includes a motor and a reducer, only a wire 12 is illustratively shown in FIGS. 1-4.

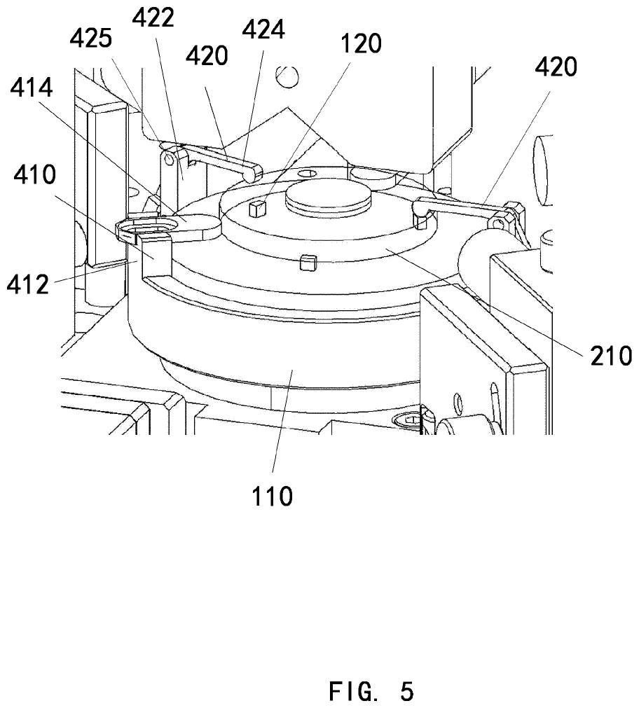

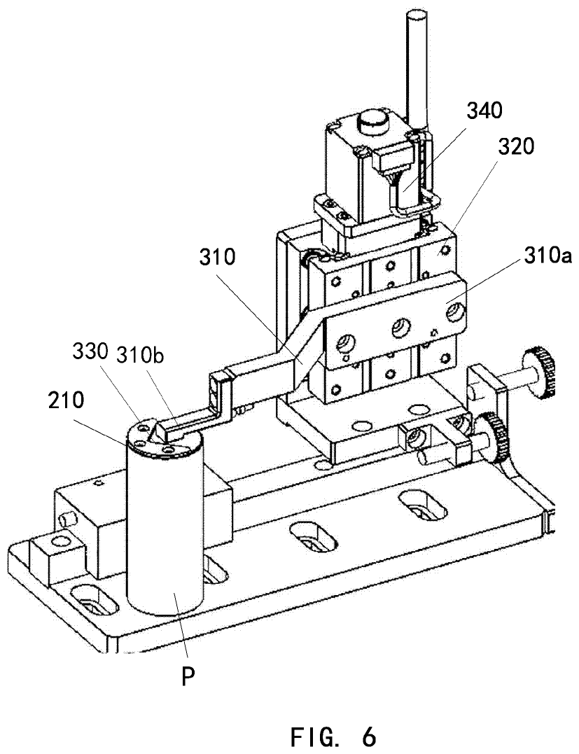

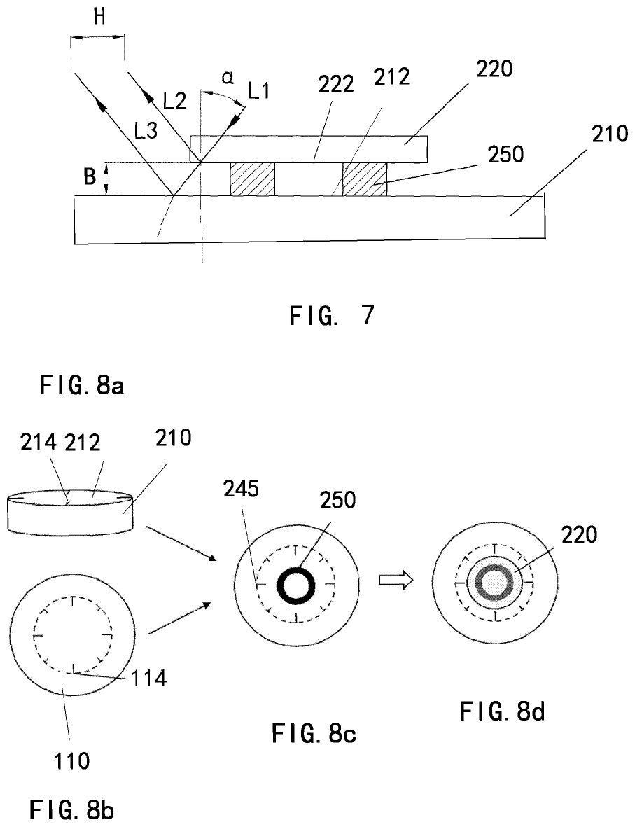

On the platform 110, a bottom substrate 210 is positioned and the paste to be monitored or measured is dispensed on it (FIGS. 2-5), and a top substrate 220 will be moved onto the paste from a storage position P, as shown in FIG. 6, by a pick-up element 310. In operation, the paste is dispensed or attached on the bottom substrate 210 firstly, the bottom substrate 210 is then be placed on the platform 110, and eventually the top substrate 220 is placed on the paste to cover it. Stubs 120 (FIG. 5) extending from the platform 110 are provided to support the bottom substrate 210 directly so that the bottom substrate 210 can be leveled in a horizontal direction by adjusting the stubs 120. Three supporting stubs 120 are provided between the platform 110 and the bottom substrate 210. Preferably, the stubs 120 can be made from an elastic material in order to minimize vibration caused to the bottom substrate 210 by the rotating platform 110. In this way, the paste can be sandwiched between a top surface 212 of the bottom substrate 210 and a bottom surface 222 of the top substrate 220, as shown in FIG. 7. First and second reflecting layers are applied to the top surface 212 of the bottom substrate 210 and the bottom surface 222 of the top substrate 220, respectively, functions of which will be described in the following. The top substrates 220, or preferably both the bottom and top substrates 210, 220, can be made from any suitable material through which a laser light can be transmitted and, in an example each of the bottom and top substrates 210, 220 can be provided as a glass sheet.

As shown in FIGS. 8a-8c, the bottom substrate 210 and the platform 110 are provided with a substrate dial 214 and a platform dial 114, respectively, and the substrate dial 214 and the platform dial 114 function as alignment features of the bottom substrate 210 and the platform 110. The bottom substrate 210 has a profile which is consistent with the platform dial 114 of the platform 110, so that the bottom substrate 210 can be precisely re-positioned in the same initial position on the platform 110 by placing it within the platform dial 114, as shown in FIG. 8c, after it has been removed from the platform 110 for other operations, for example for curing the paste in other places. The substrate dial 214 and the platform dial 114 have initially aligned lines, so that the bottom substrate 210 can be precisely re-positioned in the same orientation relative to the platform 110 after it has been removed from the platform 110. As an example, the initially aligned lines can be a 0° line in the substrate dial 214 and a 0° line in the platform dial 114, or a 90° line in the substrate dial 214 and a 90° line in the platform dial 114, or maybe a 270° line in the substrate dial 214 and a 270° line in the platform dial 114, as is indicated by a reference sign 245 in FIG. 8c.

Back to FIG. 1-5, a plurality of pushing clamps 410 and a plurality of pressing clamps 420 are provided for securing the bottom substrate 210 relative to the platform 110 in radial and circumferential directions and in the Z direction, respectively. Both the pushing clamps 410 and the pressing clamps 420 can extend from or be attached to the platform 110.

With reference to FIG. 5, the pushing clamps 410 are movable so that the bottom substrate 210 can be removed and replaced on the platform 110, and each of the pushing clamps 410 comprises a base portion 412 extending from the platform 110 and an abutting portion 414 which is configured for abutting an outer circumferential surface of the bottom substrate 210 in a substantial horizontal direction. The abutting portion 414 can be movable relative to the base portion 412 in the horizontal plane, for example, in the radial direction. Each of the pressing clamps 420 can comprise a base portion 422 extending from the platform 110 and a pressing finger 424 pivotable relative to the base portion 422 about a pivoting shaft 425 so that the bottom substrate 210 can be pressed and therefore secured on the platform 110 in the Z direction. In the embodiment, three pushing clamps 410 and three pressing clamps 420 are provided, but it should be understood that the number and the detail structure of the pushing clamps 410 and the pressing clamps 420 are not limited to those shown in the drawings.

An “alignment combination” of the alignment features with the pushing clamps 410 and the pressing clamps 420 ensures that, the bottom substrate 210, or a “paste holding combination” including the bottom substrate 210, the top substrate 220 and the paste therebetween, after being removed from the platform 110, can be precisely re-positioned on the platform 110 at the same initial position and orientation, ensuring a high consistency and precision during multiple measurement operations. For the thermal curing process and the reliability test process during which the paste holding combination needs to be moved from the platform to another place and returned onto the platform later, the provision of the alignment combination ensures that the bottom substrate has a consistent position relative to the platform.

On opposite sides of the platform 110, two rails 510 and 520 and four UV sources, for example UV lamps in the embodiment, are arranged, with two UV lamps 530 on the rail 510 and two UV lamps 540 on the rail 520, as shown in FIGS. 1-3. All the UV lamps should be arranged uniformly around the paste so that the paste can be cured evenly and the thickness of the paste can reduce evenly. In the embodiment as shown, one of the rails, 520, as well as the two UV lamps 540, is configured to be movable or removable, so that the bottom substrate 210 can be taken away. Alternatively, according to actual condition, it may be provided that only one or more UV lamp is movable along the corresponding rail or removable, or maybe two rails 510 and 520 are movable or removable, as long as the bottom substrate 210 or the paste holding combination can be removed as desired.

As described above, the top substrate 220 can be moved on the paste from a storage position P by the pick-up element 310. With reference to FIG. 6, one end 310a of the pick-up arm 310 is fixed to a supporting base 320 which in turn is fixed on the table 10, and the other end 310b of the pick-up arm 310 is provided with a suction disk 330 for picking up the top substrate 220. A numerical control unit (referred to as “NCU” hereinafter) 340 is configured for controlling operations of the pick-up arm 310 and can be provided at the supporting base 320 or some other location convenient to operate. The pick-up element 310 operates to pick up the top substrate 220 at the storage position P (FIG. 6), move it onto the paste on the bottom substrate 210, and optionally apply a downward force on the top substrate 220 to press the paste to a predetermined initial thickness value.

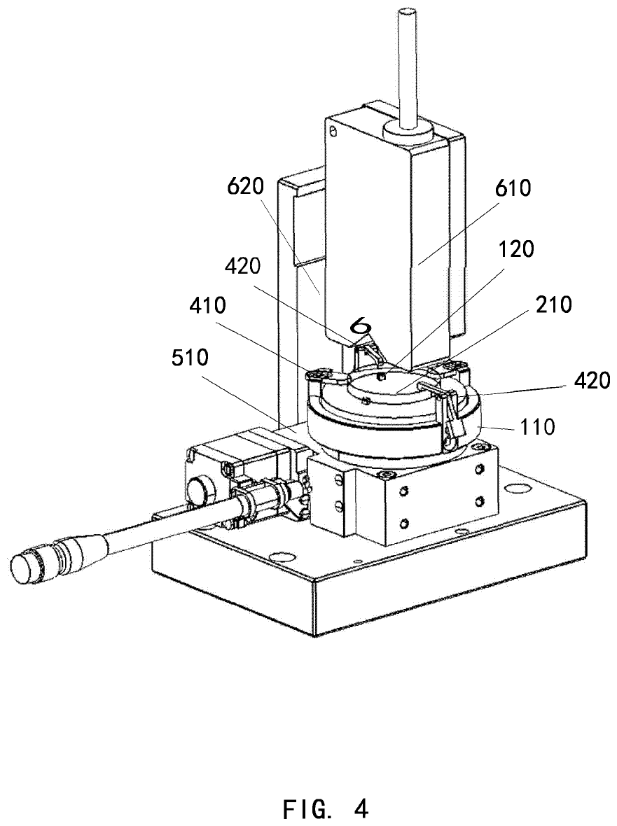

A sensor 610 is provided and attached to a post 620 which in turn is fixed to the table 10, and thus the sensor 610 is fixed relative to the table 10, as shown in FIG. 4. The sensor 610 can be a laser displacement sensor and is configured for measuring a thickness of paste at a certain point using optical principle. The thickness B of the paste can be understood as a distance from the bottom surface 222 of the top substrate 220 to the top surface 212 of the bottom surface 210, as shown in FIG. 7.

The principle how sensor 610 measures the thickness of the paste is illustrated in FIG. 7, in which the top substrate 220, the bottom substrate 210, an incident light L1 with the incident angle α, and reflected lights L2 and L3 reflected by the first reflecting layer at the bottom surface 222 of the top substrate 220 and the second reflecting layer at top surface 212 of the bottom substrate 210 are shown. The incident light L1 is emitted out from a light emitting element of the sensor 610 onto the top substrate 210 and transmitted through the top substrate 210, a portion of the incident light L1 being reflected by the first reflecting layer at the bottom surface 222 of top substrate 220, as indicated by the first reflected light L2, and others of the incident light L1 being projected onto the top surface 212 of the bottom substrate 210 without travelling through the paste and reflected by the second reflecting layer, as indicated by the second reflected lamp L3. Preferably, the top substrate 210 has a thickness which is small enough so that the two reflected lights L2 and L3 have substantially parallel reflecting directions, as shown in FIG. 7. A horizontal distance H is obtained firstly based on the reflected lamps L2 and L3 received by the light receiving element, and then the thickness B of the paste can be calculated out geometrically.

According to the disclosure, the top substrate 220 is constructed, configured or specially treated so that a laser light from the sensor 610 is allowed to be transmitted through. In an embodiment, the top substrate 220 and the bottom substrate 210 have a profile accuracy of less than ¼λ. In an embodiment, the top substrate 220 or both the top and bottom substrate are transparent glass sheets or glass sheets with a suitable coating.

In an embodiment, the top substrate 220 is further designed so that a UV light can be transmitted through, and the UV light emitted out from the UV lamps 530 and 540 are projected angledly onto a top surface of the top substrate 220 and then transmitted through the top substrate 220 into the paste to cure it. In an alternative embodiment, depending on the position of the UV lamps 530 and 540, the top substrate 220 can be further designed so that a UV light cannot be transmitted through, and the UV light emitted out from the UV lamps 530 and 540 are projected onto the paste between the top substrate 220 and the bottom substrate 210 from side.

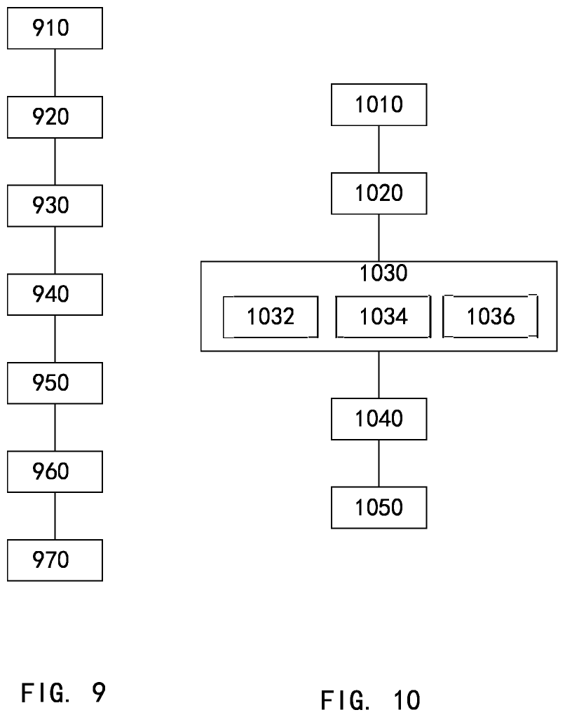

With the machine described above, a method for timely monitoring the paste shrinkage in the Z direction over UV applied time during the UV curing process includes, as shown in a flow chart in FIG. 9, a step 910, assembling and leveling the platform 110 on the table 10; a step 920, placing the bottom substrate 210 on the platform 110 with the platform dial 114 and leveling the bottom substrate 210 to be within a predetermined tolerance by adjusting the stubs 120; a step 930, securing the bottom substrate 210 on the platform using the pushing clamps 410 and the pressing clamps 420; a step 940, dispensing the paste of an adhesive sample on the bottom substrate 210 in an annular pattern, as shown in FIG. 8c; a step 950, picking up and moving the top substrate 220 from its storage position P onto the paste using the pick-up element 310; a step 960, triggering the UV lamps to cure the paste, while triggering the sensor 610; and a step 970, measuring the thickness of the paste at one point over UV applied time at predetermined time intervals while recording the thickness values.

In some embodiments, the method may further include recording the initial thickness value before step 960. Alternatively, the method may further include applying a downward force on the top substrate 220 by the pick-up element 310 to press the paste to the initial thickness value and record the initial thickness value before step 960. Still alternatively, the method may include, before step 960, rotating the platform 110 and measuring the thickness values at a preset number of points of the paste in a circumferential direction of the paste by repeating measurement operations at the predetermined time intervals and averaging the recorded thickness values to obtain an averaged thickness value as the initial thickness value and recording the initial thickness value.

The method may further include a step of making a sketch using the thickness values recorded during the curing process after the UV curing process finishes, which can reflect the thickness reduction rule of the paste over the UV applied time.

In some embodiments, the method may further include rotating the platform 110 and measuring the thickness values at the preset number of points of the paste in the circumferential direction by repeating measurement operations at the predetermined time intervals and averaging the recorded thickness values to obtain an averaged thickness value as the final thickness value, after the step 970.

Preferably, the method may further include a step of calculating the change rate, i.e. the shrink rate, of the paste after the UV curing process based on the initial thickness value in step 950, as Bᵢₙᵢ, and the final thickness value, B_{fin}, using the formula I.

As described above, the change rate of the paste in the Z direction after any process can be obtained based on the initial thickness value before the process, Bᵢₙᵢ, and the final thickness value after the process, B_{fin}, using the formula I, and, for precision consideration, it is preferable that the thickness values Bᵢₙᵢ and Bᵢₙᵢ be averaged thickness values based on many thickness values measured at one and the same set of points in the circumferential direction of the pattern of the paste before and after the certain process, respectively.

For the UV curing process, as described above, before the UV curing process and after the process finishes, the thickness values Bᵢₙᵢ and Bᵢₙᵢ can be obtained by rotating the platform 110 and measuring the thickness values at one and the same set of points in the circumferential direction and averaging the measured thickness values at these points. However, the thickness values Bᵢₙᵢ can be the predetermined thickness value which is achieved by the NCU and the pick-up element 310m instead of the averaged thickness values before the UV curing process.

A method for obtaining a change rate of a paste after a certain process includes: a preparation step 1010 in which the machine and the paste are ready for operation, a step 1020 in which the thickness values Bᵢₙᵢ is obtained by rotating the platform 110 and measuring the thickness values at a set of points in the circumferential direction of the paste and averaging the measured thickness values at these points; a step 1030 in which the certain process is carried out to the paste; and a step 1040 in which, after the certain process finishes, the thickness values B_{fin} is obtained by rotating the platform 110 and measuring the thickness values at the same set of points in the circumferential direction of the paste and averaging the measured thickness values at these points; and a step 1050 in which the change rate is calculated out based on the thickness values Bᵢₙᵢ and B_{fin} using the formula I.

The preparation step 1010 may include: the step 910, assembling and leveling the platform 110 on the table 10; the step 920, dispensing the paste of an adhesive sample on the bottom substrate 210 in an annular pattern; the step 930, placing the bottom substrate 210 on the platform 110 within the platform dial 114 and leveling the bottom substrate 210 to be within a predetermined tolerance by adjusting the stubs 120; the step 940, securing the bottom substrate 210 on the platform 110 using the pushing clamps 410 and the pressing clamps 420; the step 950, picking up and moving the top substrate 220 from its storage position P onto the paste using the pick-up element 310.

For the thermal curing process and the reliability test process in which the paste needs to be moved to other places, the step 1030 may include: a step 1032 in which the pick-up element 310 is moved away and one or more UV lamp and/or one or more rail are removed so that the paste holding combination can be removed from the platform 110; a step 1034 in which the process is carried out to the paste; and a step 1036 in which the paste holding combination is returned back to the machine and the bottom substrate 210 is secured to the platform 110 at the initial position and orientation, using the alignment features, the pushing clamps 410 and the pressing clamps 420.

Although the machine and the methods of using the machine have been described in detail as above, some examples are given for further explanation so that the technical effects and advantages can be better understood.

### Example 1

Three kinds of adhesive pastes A, N and D which are widely used in active alignment process in CCM are used in this example for comparison purpose. They are dispensed on the bottom substrate 210 in the same annular pattern as shown in FIGS. 8c-8d, with the same weight of 4.4 mg for control. The paste pattern has the same initial thickness of 150 μm which is achieved by using the pick-up element 310 to press the top surface 220 under the control of the NCU.

First of all, three pastes are subjected to the UV curing process in the machine, wherein the UV curing depths for the three pastes are 450 μm, 490 μm and 200 μm, respectively, and the UV applied time is 2 Sec, 2 Sec and 4 Sec, respectively, under UV intensiveness of 1500 mw/cm2.

The thickness values are measured and collected using the sensor 610 every 20 mSec. All the measured thickness values are plotted into sketches, as shown in FIGS. 11a-c for the three adhesives. It shows from the sketches that, for all the three adhesives, once the UV lamps and the sensor 610 are triggered the thickness value starts to decline over UV applied time, which means the UV curing agents contained in the adhesives are triggered when the pastes are exposed to the UV light and start to reaction. After the reaction of the UV curing agent completes, the thickness curves tend to be stable. By comparison, the adhesives A and N in FIGS. 11a and b have a faster UV response than D in FIG. 11d, and therefore it takes a longer time to cure the adhesive D before the thickness B becomes stable, 4 mSec. The object of timely monitoring the paste shrinkage in the Z direction is achieved.

Based on the initial thickness value of 150 μm, as Bᵢₙᵢ, and the thickness value when the thickness curves are stable, as B_{fin}, the change rate, i.e. the shrinkage rate, of the pastes can be calculated out using the formula I, as shown in FIG. 11d. FIG. 11d shows that N has a higher UV change rate than A and D. Therefore, the object of obtaining the change rate of the paste in the Z direction is also achieved.

Alternatively, for precision purpose as described above, the thickness value B_{fin} can be the averaged thickness values by rotating the platform 110 and measuring the thickness value at a set of points of the paste in the circumferential direction after the UV curing process. For example, by controlling the time interval at which the sensor 610 measures the thickness value as well as a rotation speed of the platform, 18 or 20 or any other number of points can be measured.

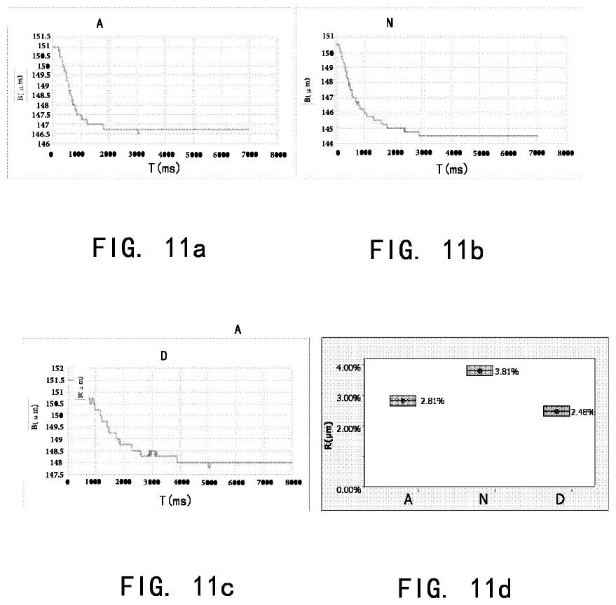

The three pastes are then subjected to the thermal curing process, and the obtained thickness value B_{fin} after the UV curing process will be the used as Bᵢₙᵢ in the thermal curing process. Then, according to the method in FIG. 10, the pick-up element 310 is removed and the paste holding combination is moved to the thermal oven to carry out the thermal curing process to the paste. Each of the three adhesive pastes A, N and D is cured at 80° C. for 1 hour. Once the thermal curing process completes, the paste holding combination is returned back to the machine according to the step 1036. The steps 1040 and 1050 are performed in which the thickness values B_{fin} is determined and the change rate is calculated out using the formula I, please refer to the FIG. 12. From FIG. 12, it can be known that, the thickness of the paste further declines during the thermal curing process, and the adhesives N and A have a higher shrinkage rate than the D adhesive.

All the steps regarding the thermal curing process are repeated for the reliability test process except the paste holding combination is moved to the box of high temperature and high humidity (HTHH) instead of the thermal oven and the humility and temperature condition during the HTHH process is different from the thermal curing condition during the thermal curing process. The pastes stay in the HTHH box with atmospheric of 85° C. & 85 rh % for 120 hrs. All the pastes absorb moisture and expand and the change rates for the three pastes after this process are shown in FIG. 13. The sketch in FIG. 13 shows that adhesive A has a higher expansion rate than adhesives D and N. It should be noted that, for the HTHH process, the change rate obtained by the formula I will be negative because the pastes expand during this process. It is easily understood that when the result of the formula I is negative, the change rate will be expansion rate and FIG. 13 shows the absolute values of the results.

In this example, with the machine and the method described in the disclosure, the paste shrinkage over UV applied time during the UV curing process in the Z direction can be timely monitored, which facilitates to analyze the shrinkage trend or the presence of the shrinkage delay during the UV curing process. In addition, this provides a great significance to understand, control or fully make use of the curing kinetic of UV curing. Resin out-gas and other factors which affect the results as listed in the background have no influence on the results with this method, and the accuracy can be ensued with the rotatable platform.

In this example, with the machine and the method described as above, the change rates of the paste in the Z direction after the UV curing process, the thermal curing process and the reliability test process are obtained. For an adhesive used in a camera, this helps to simulate a focal length change of the camera after AA process and reliability test.

### Example 2

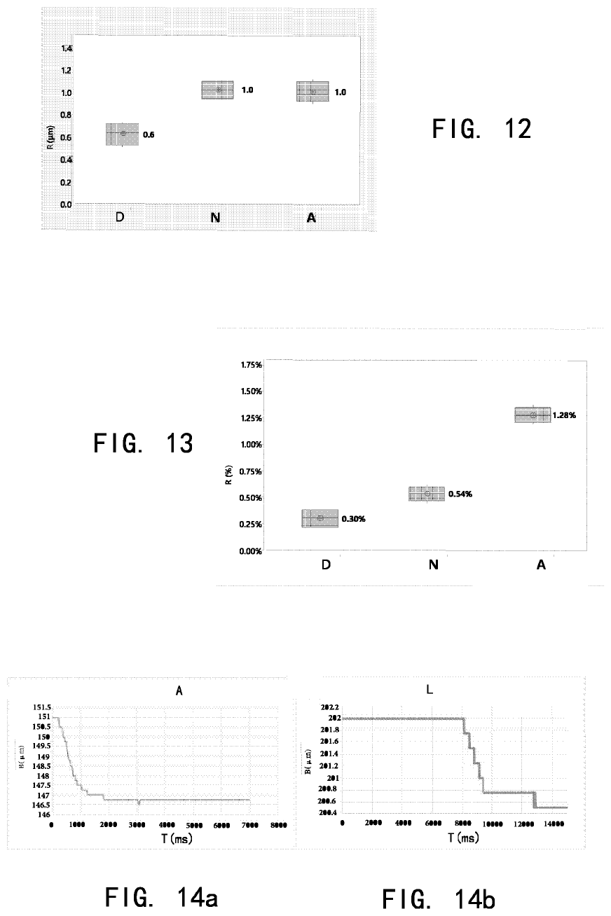

In this example, two types of adhesives A and L which contain different types of resins are used, and only the UV curing process is carried out to them. During this process, the thickness values are measured by the sensor 610 every 20 Sec and recorded accordingly, and the recorded thickness values are plotted as shown in FIGS. 14a and 14b. By comparison, it can be known that, after the UV lamps of the machine are triggered, the thickness values of the adhesive A paste decreases immediately while the thickness of the adhesive L paste substantially has no change within 8 Sec and then starts to decline, which illustrates that adhesive L has a UV delay which may be due to the inclusion of some special composition, for example, cationic epoxy curing agent.

Therefore, in addition to timely monitor the paste shrinkage in the Z direction during the UV curing process, the paste's UV response performance for difference compositions, for example, the type or performance of the UV curing agent contained in the adhesive, can be identified or determined using the machine and the method described above.

Although the present disclosure as well as two examples have been shown and described, it would be appreciated by those skilled in the art that changes may be made in these embodiments without departing from the principles and spirit of the disclosure, the scope of which is defined in the claims and their equivalents.

What is claimed is:

1. A machine of paste shrinkage and expansion for timely monitoring a paste shrinkage in a Z direction during a UV curing process, the machine comprising:
a bottom substrate on which the paste to be monitored is dispensed;
a top substrate which is configured to be placed on the paste so that the paste is sandwiched between a top surface of the bottom substrate and a bottom surface of the top substrate, wherein the top surface of the bottom substrate and the bottom surface of the top substrate are provided with first and second reflecting layers, respectively, and define a thickness of the paste;
a platform for supporting the bottom substrate; a plurality of pressing clamps configured for securing the bottom substrate on the platform in the Z direction;
a plurality of pushing clamps configured for securing the bottom substrate in radial and circumferential directions;
a plurality of UV sources positioned uniformly around the paste; and
a sensor configured for measuring the thickness of the paste.

2. The machine of paste shrinkage and expansion according to claim 1, wherein the platform is rotatable.

3. The machine of paste shrinkage and expansion according to claim 2, wherein the bottom substrate and the platform comprise a substrate dial and a platform dial, respectively, and the bottom substrate has a profile consistent with the platform dial.

4. The machine of paste shrinkage and expansion according to claim 1, further comprising a pick-up element configured for moving the top substrate from a storage position onto the paste which is dispensed on the bottom substrate in advance.

5. The machine of paste shrinkage and expansion according to claim 4, wherein the pick-up element is further configured for pressing the paste in the Z direction.

6. The machine of paste shrinkage and expansion according to claim 5, further comprise a NCU for controlling operations of the pick-up element so that the pick-up element presses the paste to a predetermined initial thickness value.

7. The machine of paste shrinkage and expansion according to claim 1, further comprise two rails, wherein the plurality of UV sources are four UV lamps and two UV lamps are fixed on each of the two rails, and one or two of the rails and/or one or more UV lamp are movable.

8. The machine of paste shrinkage and expansion according to claim 1, wherein the sensor comprises a light emitting element for emitting an incident light onto the top bottom, and a light receiving element for receiving reflected lights reflected by the top surface of the bottom substrate and the bottom surface of the top surface, the sensor being configured for obtain the thickness of the paste based on the received reflected lights geometrically.

9. The machine of paste shrinkage and expansion according to claim 1, wherein the bottom substrate and the top substrate are configured as glass sheets.

10. The machine of paste shrinkage and expansion according to claim 1, wherein a plurality of stubs extend from the platform to support the bottom substrate and are configured for level the bottom substrate.

11. A method for timely monitoring a paste shrinkage in a Z direction during a UV curing process using the machine of paste shrinkage and expansion according to claim 1, wherein the method includes the steps of:
placing the bottom substrate on the platform and leveling the bottom substrate;
securing the bottom substrate on the platform using the pushing clamps and the pressing clamps;
dispensing the paste on the bottom substrate in an annular pattern;
picking up and moving the top substrate onto the paste;
triggering the UV lamps, starting to cure the paste, while triggering the sensor; and
measuring the thickness values of the paste at one point over UV applied time at predetermined time intervals while recording the thickness values, until the UV curing process completes.

12. The method according to claim 11, further including, before triggering the UV lamps:
measuring the thickness of the paste and recording it as an initial thickness value; or
applying a downward force on the top substrate to press the paste to a predetermined initial thickness value and recording it; or
rotating the platform, measuring the thickness values at a predetermined set of points of the paste in a circumferential direction of the paste, and averaging the recorded thickness values to obtain an averaged thickness value as the initial thickness value.

13. The method according to claim 12, further including a step of making a sketch using the thickness values measured and recorded over UV applied time.

14. The method according to claim 12, further including:
rotating the platform;
measuring the thickness values at the preset set of points of the paste in the circumferential direction;
averaging the recorded thickness values to obtain an averaged thickness value as the final thickness value; and
calculating the change rate, R, of the paste in the Z direction after the UV curing process based on the initial thickness value, as Bᵢₙᵢ, and the final thickness value, B_{fin}, using the formula I: R=(B_{fin}−Bᵢₙᵢ)/B_{fin}.

15. A machine of paste shrinkage and expansion for obtaining a change rate of a paste in a Z direction after a certain process, wherein the machine comprises:
a bottom substrate on which the paste to be monitored is dispensed;
a top substrate which is transparent and configured to be placed on the paste so that the paste is sandwiched between a top surface of the bottom substrate and a bottom surface of the top substrate, wherein the top surface of the bottom substrate and the bottom surface of the top substrate are provided with first and second reflecting layers, respectively, and define a thickness of the paste, and wherein the bottom substrate and the platform comprise a substrate dial and a platform dial, respectively, and the bottom substrate has a profile consistent with the platform dial;
a rotatable platform for supporting the bottom substrate;
a plurality of pressing clamps configured for securing the bottom substrate on the platform in the Z direction;
a plurality of pushing clamps configured for securing the bottom substrate on the platform in radial and circumferential directions; and
a sensor configured for measuring the thickness of the paste.

16. A method for obtaining a change rate of a paste in a Z direction after a certain process using the machine of paste shrinkage and expansion according to claim 15, wherein the method includes:
placing the bottom substrate on the platform within the platform dial and leveling the bottom substrate;
securing the bottom substrate on the platform using the pushing clamps and the pressing clamps;
dispensing the paste on the bottom substrate in an annular pattern;
picking up and moving the top substrate onto the paste;
obtaining an initial thickness value, Bᵢₙᵢ;
carrying out the certain process to the paste; and
obtaining a final thickness value, B_{fin}, by rotating the platform and measuring the thickness values at the set of points and averaging the measured thickness values at these points; and
calculating out the change rate based on the initial thickness value and final thickness value using the formula I: R=(Bᵢₙᵢ−B_{fin})/Bᵢₙᵢ.

17. The method according to claim 16, wherein the certain process is a UV curing process which is carried out in the machine, and the initial thickness value is achieved by pressing the top substrate with the pick-up element.

18. The method according to claim 16, wherein the certain process is a thermal curing process or a reliability test process, and the initial thickness value is achieved by rotating the platform and measuring the thickness values at a predetermined set of points in the circumferential direction of the paste and averaging the measured thickness values at these points.

19. The method according to claim 18, wherein the method includes: removing a paste holding combination including the bottom substrate, the top substrate and the paste therebetween before the certain process is carried out and returning the paste holding combination back onto the platform at the initial position and orientation after the certain process is carried out.
